(19) United States
(12) Patent Application Publication
(10) Pub. No.: US 2011/0054391 A1
(43) Pub. Date: Mar. 3, 2011

(54) ANALYTE SENSING AND RESPONSE SYSTEM

(76) Inventors: W. Kenneth Ward, Portland, OR (US); Jessica R. Castle, Portland, OR (US); Julia Engle, Aurora, CO (US)

(21) Appl. No.: 12/793,632

(22) Filed: Jun. 3, 2010

Related U.S. Application Data
(63) Continuation-in-part of application No. 11/592,034, filed on Nov. 1, 2006.
(60) Provisional application No. 60/834,279, filed on Jul. 28, 2006.

(51) Int. Cl. A61M 5/00

(52) U.S. Cl. 604/66

(57) ABSTRACT

Electrochemical systems for measuring an analyte concentration, and correcting any surplus or deficiency in the measured concentration. More specifically, systems for measuring an analyte level in a fluid with an implantable sensor, processing the measurements with a front-loaded delivery algorithm having a fluid delivery period and a refractory period, and determining an appropriate fluid infusion rate in response to the measurements.

### CROSS REFERENCE TO RELATED APPLICATION

This application is a continuation-in-part of U.S. patent application Ser. No. 11/592,034, filed Nov. 1, 2006 and entitled ANALYTE SENSING AND RESPONSE SYSTEM, which application claims priority under 35 U.S.C. §119(e) and all applicable international law to U.S. Provisional Patent Application Ser. No. 60/834,279 filed Jul. 28, 2006, which are both incorporated herein by reference in their entirety for all purposes, and further claims priority under 35 U.S.C. §119(e) and all applicable international law to U.S. Provisional Patent Application Ser. No. 61/183,807 filed Jun. 3, 2009, which is incorporated herein by reference in its entirety for all purposes.

### FIELD OF THE INVENTION

The invention generally relates to electrochemical systems for measuring an analyte concentration and correcting any surplus or deficiency in the measured concentration. More specifically, the invention relates to systems for measuring an analyte level in a fluid with an implantable sensor, processing the measurements with an algorithm, and determining an appropriate fluid infusion rate in response to the measurements.

### BACKGROUND

Maintaining appropriate analyte levels in the bloodstream of mammals, including humans, is extremely important, and failure to do so can lead to serious health problems and even death. For example, in diabetic patients, malfunction of the pancreas can lead to uncontrolled blood glucose levels, possibly resulting in hypoglycemic or hyperglycemic shock. To compensate for this and to maintain an appropriate blood glucose level, diabetics must receive timely and correct doses of insulin. Similarly, many other analytes commonly are measured in the blood of humans and in other fluids, for the purpose of determining an appropriate response to any measured surplus or deficiency of the analyte.

One method of measuring an analyte concentration in the blood of a mammal is to use an implantable sensor to measure the concentration, and a number of previous patented inventions relate to various aspects of such sensors. U.S. Pat. No. 5,711,861 to Ward et al. claims a disc-shaped sensor device having multiple anode/cathode pairs of electrodes for taking redundant analyte measurements. U.S. Pat. No. 6,212,416 to Ward et al. adds a coating to the sensor to inhibit formation of collagen or to enhance the sensitivity of the sensor in the presence of the analyte, and claims multiple redundant sensors (as opposed to a single sensor with multiple electrode pairs). U.S. Pat. No. 6,466,810 to Ward et al. claims a sensor with a single cathode and a plurality of anodes on each side, to provide redundant measurements without requiring multiple cathodes.

Once analyte measurements have been obtained with a sensor—whether the sensor is implantable or otherwise—a response often must be determined, typically in the form of a fluid infusion rate to alter the analyte concentration to a more desirable level. The infused fluid may contain the analyte itself, or it may contain a substance the presence of which affects the analyte level. For example, if the measured analyte is glucose, the infused fluid may contain glucose, or it may contain insulin.

Typically, an algorithm is used to determine a fluid infusion rate from analyte measurements, and several such algorithms are known. For example, a glucose-controlled insulin infusion system incorporating a proportional derivative (PD) method is disclosed in U.S. Pat. No. 4,151,845 to Clemens. U.S. Pat. No. 6,558,351 to Steil et al. claims an insulin infusion system using a proportional integral derivative (PID) algorithm that takes a patient's history of glucose levels into account when determining the infusion rate, by integrating the difference between the measured glucose level and the desired glucose level from some prior time up to the present. U.S. Pat. No. 6,740,072 to Starkweather et al. adjusts the parameters of the insulin infusion algorithm dynamically in response to exercise, sleep, and other external events.

However, despite the use of various algorithms to determine a response to a measured analyte concentration, no algorithm has been developed that takes into account both current and prior analyte levels in a manner that adequately reflects the dynamic nature of the measured concentration. In the case of glucose measurements and insulin infusion, for example, none of the previously developed algorithms are able to simulate completely the normal insulin response of a healthy pancreas. Thus, a need exists for an improved system for measuring an analyte concentration, processing the measurements using an algorithm that adequately takes into account the dynamics of the analyte, and determining a response.

Additionally, when insulin is infused in the typical manner (administered under the skin, i.e. in the subcutaneous space), even so-called “fast” insulins (e.g., aspart insulin, lis-pro insulin, and glulisine insulin) are relatively slow in their action compared to naturally occurring insulin action in non-diabetic humans. All three insulin types, aspart insulin, lis-pro insulin and gluisine insulin, are much slower in terms of onset and offset as compared to naturally occurring insulin that non-diabetic humans make in their pancreatic beta cells and secrete into their portal circulation. During control of diabetes, it is quite common to have the glucose level fall too low, due in large part to the types of insulin that are currently available on the market.

Because of the slow response time of currently-available insulin, it can persist in the human body for up to 8 hours, which is many times longer than the duration of non-diabetic humans endogenously-secreted insulin. This prolonged persistence of insulin often can lead to hypoglycemia that can occur several (e.g., 2 to 4.5) hours after meals. Hypoglycemia can be a very severe event, leading to seizures, coma, and even death Milder responses to hypoglycemia can also occur, such as social embarrassment and temporary loss of judgment. When hypoglycemia is severe and recurring, it can cause permanent loss of memory and judgment. Thus, a need exists for a solution to the current problems experienced with insulin administration to diabetics.

### BRIEF DESCRIPTION OF THE DRAWINGS

FIG. 1 is a partially cut-away perspective view of an analyte sensor.

FIG. 2 is a cross-sectional view of the sensor shown in FIG. 1.

FIG. 3 is a schematic flow chart of an analyte monitoring system, including an analyte sensor, electronics, telemetry, and computing components.

FIG. 3A is a schematic drawing of a system for sensing analyte levels and delivering an appropriate amount of a modulating substance.

FIG. 4 is a graph illustrating results of a closed loop insulin infusion experiment in rats.

FIG. 5 is a table comparing glucose levels in rats before and after insulin infusion.

FIG. 6 shows a graph plotting glucose oscillations versus time in a single animal.

FIG. 7 is a graph showing a gain schedule zone diagram.

FIG. 8 is a graph showing pancreatic response profiles using three different algorithms.

FIG. 9 is a graph showing glucose oscillations and glucagon infusion rate versus time using an algorithm having a low proportional gain factor and a low derivative gain factor.

FIG. 10 is a graph showing glucose oscillations and glucagon infusion rate versus time using an algorithm having a high proportional gain factor and a high derivative gain factor and a refractory period.

FIG. 11 is a graph showing glucose oscillations and glucagon infusion rate versus time using an algorithm having a low proportional gain factor and a low derivative gain factor.

FIG. 12 is a graph showing glucose oscillations and glucagon infusion rate versus time using an algorithm having a high proportional gain factor and a high derivative gain factor and a refractory period.

FIG. 13 is a chart showing daytime results—percentage of time in target range for subjects receiving no glucagon, low gain glucagon, and front-loaded glucagon.

FIG. 14 is a chart showing percentage of time in hyperglycemic range for subjects receiving no glucagon, low gain glucagon, and front-loaded glucagon.

FIG. 15 is a chart showing time in hypoglycemic range for subjects receiving no glucagon, low gain glucagon, and front-loaded glucagon.

FIG. 16 is a chart showing prevention of hypoglycemia for subjects receiving no glucagon, low gain glucagon, and front-loaded glucagon.

FIG. 17 is a graph showing glucose oscillations, insulin delivery rate and glucagon infusion rate versus time.

FIG. 18A is a graph showing glucose oscillations, insulin delivery rate, and meals versus time

FIG. 18B is a graph showing glucose oscillations, insulin delivery rate, meals and glucagon infusion rate versus time.

### DESCRIPTION

The present disclosure generally relates to systems for measuring an analyte level in a fluid with an implantable sensor, processing the measurements with an algorithm, and determining an appropriate fluid infusion rate in response to the measurements. The disclosed sensors generally are suitable for implantation into a mammal, and may include various features such as multiple anode/cathode pairs of electrodes for taking redundant glucose measurements, coatings to inhibit formation of collagen or to enhance the sensitivity of the sensor in the presence of glucose, and/or a single cathode with a plurality of anodes on each side, to provide redundant signals without requiring multiple cathodes.

The disclosed algorithms may use current and previous analyte values, and current and previous analyte rates of change, to determine an appropriate fluid infusion rate in response. These algorithms weigh more recent analyte values and analyte rates of change more heavily than more remote values and rates of change. This disclosure refers to an algorithm having these characteristics as a “Fading Memory Proportional Derivative” (FMPD) algorithm.

Additionally and/or alternatively, the disclosed algorithms may be configured to result in a specific pattern of fluid administration, wherein fluid is infused as a time-limited and/or amount limited pulse, also referred to as a period, dose and/or bolus, followed by a refractory period. The refractory period is defined as the period of time, after the delivery of a pulse of fluid, during which the algorithm does not allow further administration of fluid. The term “front-loaded fluid delivery” may be used to describe the pattern of brisk doses of fluid, with each dose being followed by a refractory period.

### I. IMPLANTABLE SENSORS AND ANALYTE MONITORING SYSTEMS

This section describes a particular embodiment of an analyte sensor suitable for use with the present invention, and a commensurate monitoring system embodiment suitable for use with the disclosed sensor.

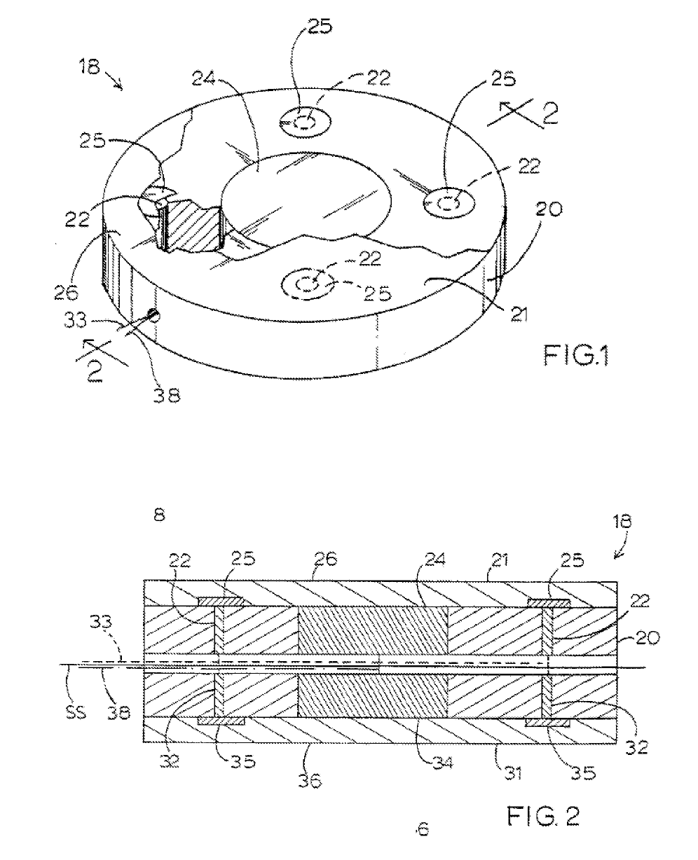

FIGS. 1 and 2 illustrate a disc-shaped glucose sensor having two opposing faces, each of which has an identical electrode configuration. Alternatively, a disc-shaped sensor may be used in which an electrode configuration is provided on only one side of the sensor. One of the faces can be seen in the partially cut-away perspective view in FIG. 1. Sensor 18 includes a disc-shaped body 20. On planar face 21 of sensor 18, four platinum anodes 22 are symmetrically arranged around a centrally disposed silver chloride cathode 24. Each anode 22 is covered by an enzyme layer 25 including the active enzyme glucose oxidase and stabilizing compounds such as glutaraldehyde and bovine serum albumin (BSA). A semi-permeable membrane layer 26 covers all of the electrodes and individual enzyme layers. The thickness and porosity of membrane layer 26 is carefully controlled so as to limit diffusion and/or transport of the analyte of interest (in this embodiment, glucose) from the surrounding fluid into the anode sensing regions. The mechanism of selective transport of the analyte of interest through the membrane may involve one or more of the following principles: molecular size exclusion, simple mass transfer, surface tension phenomena, or chemically mediated processes.

FIG. 2 shows a cross-section of sensor 18. Sensor 18 has a plane of symmetry SS, which is normal to the plane of the figure. Under face 31 of sensor 18, anodes 32 are spaced equidistantly apart from cathode 34. Enzyme layers 35 cover anodes 32. A semi-permeable polyurethane membrane 36 covers the enzyme layers and electrodes. Each of anodes 22 and 32 are connected to a common anode wire 33 that leads out of the sensor for electrical connection to an electrometer. Similarly each of cathodes 24 and 34 are connected to a common cathode lead 38, which leads out of sensor 18 for electrical connection to the electrometer.

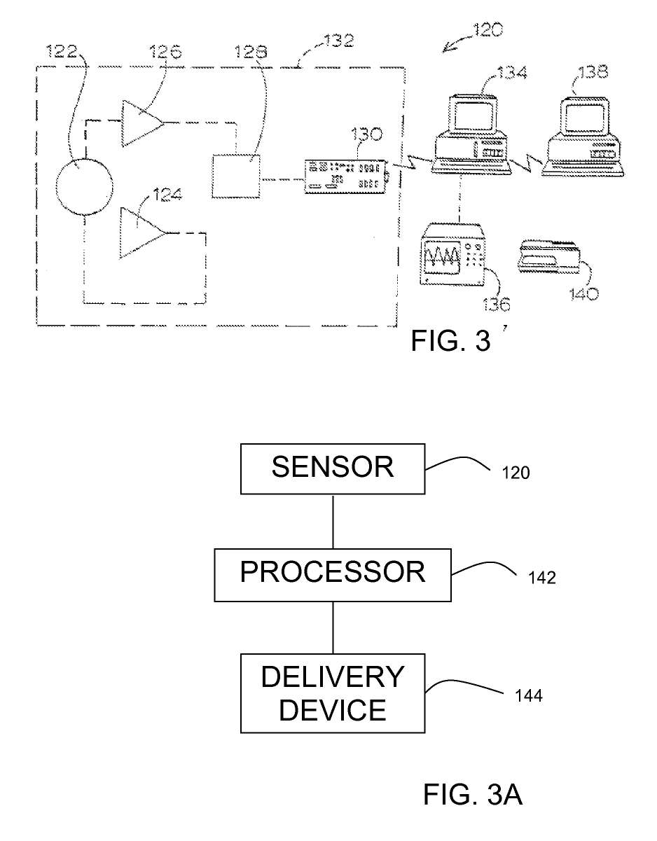

FIG. 3 shows schematically how an implantable analyte sensor (in this embodiment, a glucose sensor) may be connected in a glucose monitoring system 120. Electrodes in sensor 122 are polarized by polarizing circuit 124.

Sensor 122 is connected to electrometer 126, which is configured to sense small changes in electric current, and to translate electric current measurements into voltage signals. Voltage signals from electrometer 126 are telemetry conditioned at 128, and conveyed to a transmitter 130 for radio transmission. All of the components within box 132 may be implanted into the patient as a single unit.

Externally, radio signals from transmitter 130, which in this embodiment are indicative of glucose concentrations in the patient's blood, are transmitted to a receiver 134. Receiver 134 may be connected to a monitor 136 for data monitoring. The same receiver computer, or another computer 138, may be used to analyze the raw data and to generate glucose concentration information. A printer 140 may be connected to computer 138 and configured to generate hard copies of the analyzed data.

FIG. 3A shows a flow chart illustrating a system for correcting analyte concentrations in a mammal. Sensor 120 is a sensor configured to detect electrochemical characteristics in a bodily fluid such as blood, indicative of analyte concentration. For example, sensor 120 may use an enzyme such as glucose oxidase to detect changes in glucose concentration. Alternatively, sensor 120 may use enzymes such as cholesterol oxidase or other enzymes to detect concentrations of other analytes.

Sensor 120 transmits data to processor 142. Processor 142 uses a fading memory algorithm to calculate an appropriate response such as an amount of insulin to deliver for normalizing an abnormal glucose concentration.

Processor 42 communicates instructions to delivery device 144 resulting in delivery of the corrective substance to the patient. Any one, two, or all of the components including sensor 120, processor 142, and delivery device 144 may be positioned inside or outside the patient.

### II. FMPD ALGORITHMS

This section describes a novel algorithm that may be used to analyze data indicative of analyte concentration—such as data obtained with the sensor system described in Section I above—and to determine a response. Typically, the response will take the form of a fluid infused into the patient's blood, into the subcutaneous tissue or into the peritoneal space, to compensate for any surplus or deficiency in the measured analyte concentration. In this section, the following additional abbreviations and definitions will be used:
PE=proportional error=the difference between a measured analyte value and a desired analyte value
DE=derivative error=deviation of the rate of change of an analyte value from zero
K_{PE}=gain coefficient for proportional error
K_{DE}=gain coefficient for derivative error
W_{PE}=weight given to a proportional error
W_{DE}=weight given to a derivative error
PE′=weighted proportional error term that includes the gain coefficient factor (see equation 2 below)
DE′=weighted derivative error term that includes the gain coefficient factor (see equation 3 below)
Z_{PE}=historical steepness coefficient for proportional error.
Z_{DE}=historical steepness coefficient for derivative error
t=time, measured backward from the present (i.e., t=10 indicates 10 minutes back into history
R=analyte infusion rate
   In terms of these definitions, the fundamental equation utilized by the FMPD algorithm is:

$$R = \frac{1}{n} {\sum }_{i = 1}^{n} \{ {PE}^{′} ( {t}_{i} ) + {DE}^{′} ( {t}_{i} ) \} \qquad ( 1 )$$

where each sum is over any desired number n of discrete analyte measurements (also known as history segments), and where the terms in each sum are defined at each particular time t, by:

PE′(tᵢ)=K_{PE}×W_{PE}(tᵢ)×PE(tᵢ) (2),

DE′(tᵢ)=K_{DE}×W_{DE}(tᵢ)×DE(tᵢ) (3).

The weight factors W_{PE} and W_{DE} in equations (2) and (3) generally may be any factors that decrease with increasing time (measured backward from the present), so that contributions to the sum in equation (1) are weighted less heavily at more remote times. In one embodiment, these factors are defined as decaying exponential functions:

W_{PE}(t)=e^{-Z}^{_{PE}}^{t} (4),

W_{DE}(t)=e^{-Z}^{_{DE}}^{t} (5).

The normalizing factor 1/n is provided in equation (1) to compensate for the fact that making the measurement interval smaller (say, every one minute instead of every 5 or 10 minutes) will increase the number of terms, and thus make the sum of the weighted terms in equation (1) larger. However, in an alternate embodiment, this factor may equivalently be incorporated into any of the elements appearing in equations (2) or (3), so that its appearance in equation (1) is somewhat arbitrary.

In the embodiment represented by equations (4) and (5), the values of the steepness coefficients Z_{PE} and Z_{DE} determine the rate of exponential decay of the weight factors, and thus, along with the values of the gain coefficients K_{PE} and K_{DE}, determine the relative weights of the various terms in the sum of equation (1). Thus, by varying the magnitudes of Z_{PE} and Z_{DE}, one can vary the degree to which the history of analyte values—in the form of the proportional error and the derivative error—are utilized. More specifically, smaller values of Z_{PE} and Z_{DE} result in a more slowly decaying weight function, so that the past history of the analyte's behavior being taken into greater consideration, whereas larger values of Z_{PE} and Z_{DE} result in a more rapidly decaying weight function, so that the past history is given less weight.

The values of the steepness coefficients Z_{PE} and Z_{DE} relative to each other also may be adjusted to change the relative importance of the history of the proportional error versus the history of the derivative error. For example, if Z_{DE} is chosen to be larger than Z_{PE}, the derivative error weight will decay more rapidly than the proportional error weight, so that less of the history of the derivative error will be taken into account in comparison to the history of the proportional error. Conversely, by choosing Z_{PE} larger than the Z_{DE}, less of the history of the proportional error will be taken into account in comparison to the history of the derivative error.

Other embodiments of an FMPD algorithm may display similar, but non-exponential behavior. For example, the weights of the terms in equation (1) may decrease linearly or polynomically backward in time, rather than exponentially. Generally, FMPD algorithms are characterized by the time-dependent weight of the terms that determine the rate of fluid infusion—with more remote terms being weighed less heavily than more recent terms—rather than by the precise functional dependence of those terms on time.

The values of the gain coefficients K_{PE} and K_{DE} affect the overall weight of the proportional error relative to the weight of the derivative error, irrespective of the values of the steepness coefficients Z_{PE} and Z_{DE}. Thus, a large value of K_{PE} relative to the value of K_{DE} leads to a greater weighting of all of the proportional errors compared to the derivative errors, independent of the manner in which the weights of the various errors change over time.

### III. EXAMPLES

This section describes several examples of systems using analyte sensor systems and/or FMPD algorithms such as those described in Sections I and II above.

### Example 1

### Animal Trials

This example describes the use of an FMPD algorithm in a trial involving laboratory rodents. Using Labview 6.1 software (National Instruments Inc, Austin Tex.), a software package was developed that implemented the FMPD algorithm. In the developed package, the main parameters of the algorithm are adjustable by the subject (e.g., the user). Measured glucose levels are entered into the program manually to compute the prescribed insulin dose. The program also contained a feature for running simulations based on data entered in a text file. In one embodiment of the FMPD algorithm, the values of the coefficients of the algorithm were set as follows:

K_{PE}=0.00015

K_{DE}=0.025

Z_{PE}=0.025

Z_{DE}=5

In order to create a model of Type 1 diabetes, Sprague-Dawley rats (Charles River Labs, Charles River, Mass., 01887) weighing 300-500 grams were given 200 mg/kg of alloxan. Only animals whose subsequent blood ketone values were greater than 1.5 mM (i.e. those considered to have Type 1 diabetes) were included in the study. Animals were treated every day with one or two subcutaneous injections of Lantus (Insulin Glargine, Aventis, Bridgewater, N.J., 08807) and/or Regular insulin (Novo-Nordisk, Copenhagen).

Rats underwent closed loop studies for six hours while on a homeothermic blanket (Harvard Apparatus, Holliston, Mass.) under anesthesia (1.5-2.5% isoflurane with 40% oxygen and 1 L/min medical air). Venous access was created by placing a 26 g catheter in the saphenous vein. The tip of the animal's tail was nicked to measure blood glucose concentration every five minutes throughout the study. Measurements were made with two hand held glucose meters (Sure Step, Johnson & Johnson Lifescan, Milipitas, Calif., 95035; AccuChek, Roche Diagnostics, Indianapolis, Ind., 46038), the mean value of which was used to calculate the insulin infusion rate. A thirty minute baseline preceded initiation of the FMPD algorithm-based insulin infusion. The insulin was diluted, one unit of Regular insulin (Novo, Copenhagen) per one ml saline. The diluted insulin was placed in a syringe pump (PHD 2000, Harvard Apparatus, Holliston, Mass.) and infused into the saphenous vein catheter. The mean of the blood glucose at each five minute reading was entered into the FMPD algorithm. Insulin infusion rates calculated by the FMPD were followed for the final five-and-a-half hours of the study.

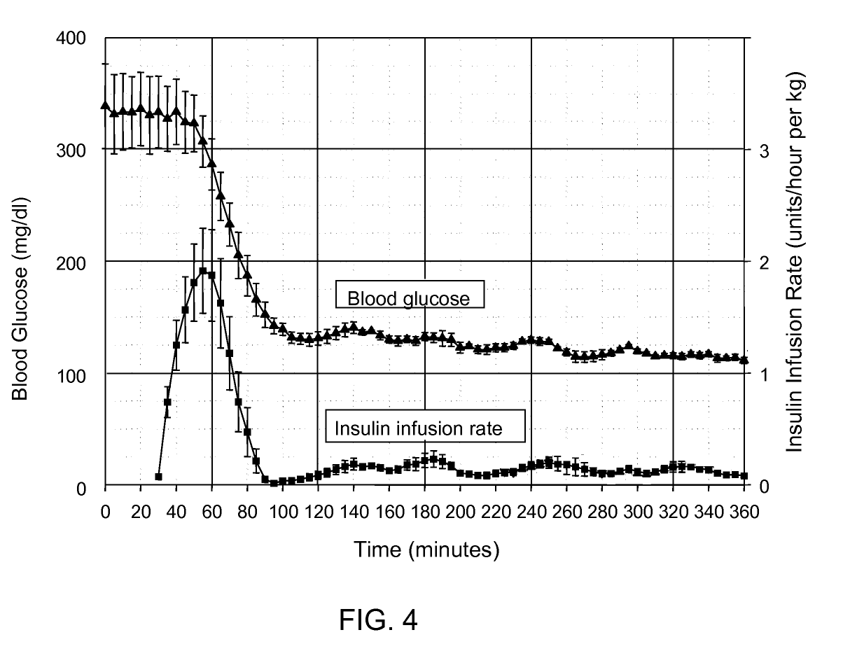

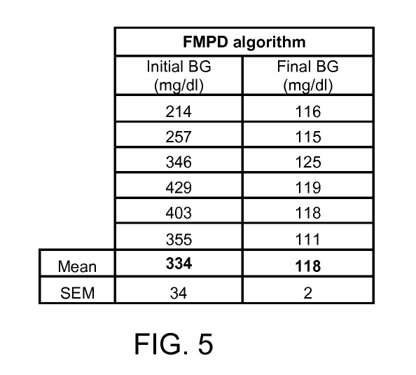

In addition to steady state assessments, dynamic aspects of closed loop control were also examined. These aspects included the oscillations of glucose level during the final 240 minutes of the closed loop control study. After identification of peaks and valleys, we then calculated the frequencies of oscillations in all studies and examined the degree to which the oscillations were convergent (decreasing amplitudes over time) or divergent (increasing amplitudes). During closed loop control, in an ideal situation, oscillations of glucose should be of small amplitude and should not increase over time. Students' unpaired t tests were used for comparisons, and data are presented as mean±SEM. A level of 0.05 was used as the criterion for significance. Results of closed loop studies are shown in FIG. 4, which portrays mean (+standard error of the mean) data for blood glucose measurements and insulin infusion rates for 6 diabetic rats. The FMPD algorithm was used to control blood glucose in these animals. A low rate basal insulin infusion was given such that insulin infusion persisted even when glucose was slightly lower than the set-point of 100 mg/dl. See also FIG. 5, which shows that the final glucose level was much lower than the initial glucose level in all animals. The final glucose level averaged 118+2.0 mg/dl (minutes 240-360).

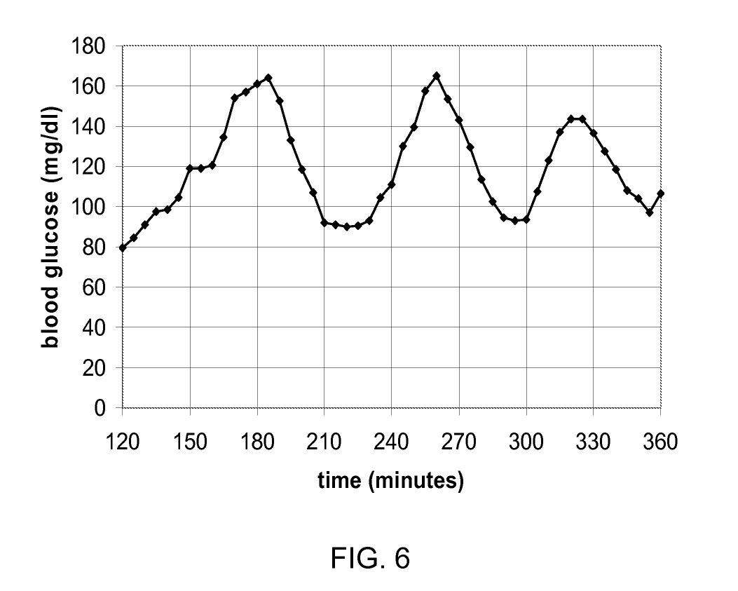

The amplitude of oscillations (distance from peak to valley) averaged 10.7+2.9%. In terms of assessing whether oscillations converged or diverged over the course of the final 4 hours of the study, we compared amplitude data during early control in minutes 120-235 with later control in minutes 240-360. An example in one animal of the oscillations during the final four hours of the study is shown in FIG. 6. There was a tendency for glucose values to converge, rather than to diverge during closed loop control. The oscillation frequency averaged 0.79 cycles per hour.

By analysis if the foregoing results, it can be understood that the FMPD algorithm is a novel closed loop insulin control algorithm that utilizes a time-related weighting of proportional and derivative glucose data. The weighting function can be thought of as a fading memory of glucose levels and trends, and is based on the fact that the islet's physiological response to glucose utilizes current information and a fading memory of previous information. Animal studies showed that blood glucose was very well controlled during the closed loop control studies. This comparison demonstrates that, in the setting of venous glucose sampling and venous insulin delivery, this method enhances glucose control without causing undue hypoglycemia. In the situation in which there is a greater efferent delay or greater afferent delay, a less aggressive approach (using lower gain parameters) may be necessary to minimize the risk of hypoglycemia.

### Example 2

### Gain Scheduling

One of the potential problems with a closed loop system of glucose control is that there can be a delay of the action of the infused insulin. For example, if one gives insulin by subcutaneous infusion, its action is much slower (due to the need for insulin to be absorbed before its action can be exerted) than if one gives the insulin intravenously. When a delay exists, it raises the possibility of overcorrection hypoglycemia. For example, assume that glucose is rising and that accordingly, the calculated insulin infusion rate also rises. However, let us assume that there is also a delay in the action of insulin in terms of its effect to reduce glucose level. The potential problem is that the algorithm will continue to increase the insulin infusion rate and that by the time the insulin finally acts, there will be a great deal of insulin that has been administered. Accordingly, the glucose can fall to very low values, a problem termed overcorrection hypoglycemia.

One method of reducing the chance of experiencing overcorrection hypoglycemia is to rapidly reduce (or discontinue) the insulin infusion rate as soon as glucose begins to fall (or even as soon as the rate of rise of glucose declines towards zero). Such adjustments can be termed gain scheduling, that is making an adjustment during the algorithm utilization based on results obtained during closed loop control.

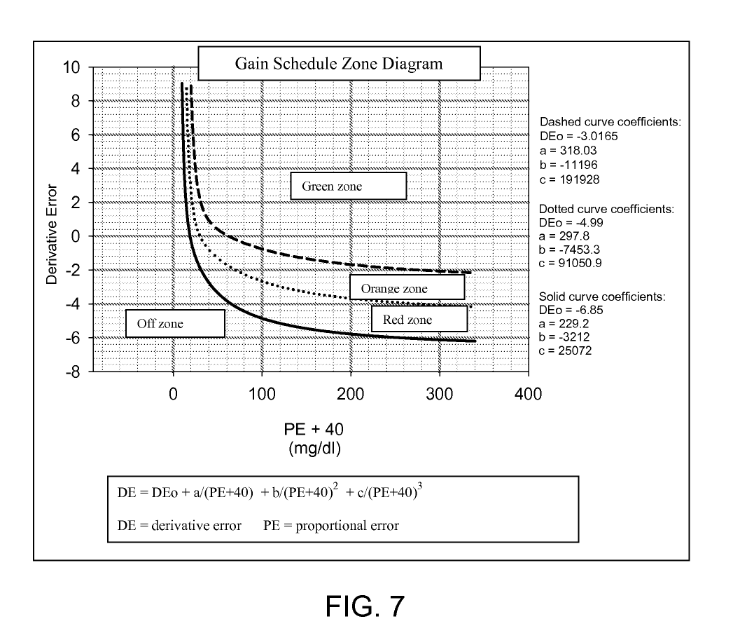

Gain scheduling is a method that can be included in many types of algorithms. Gain scheduling essentially adjusts the gain parameters of the control algorithm such that desired responses can be obtained according to different ongoing results. Gain parameters are adjusted based on decision rules that utilize functions of input and output parameters. When considering the extremes of blood glucose levels, hypoglycemia is acutely more serious than hyperglycemia. If hypoglycemia is severe enough, coma, seizures or death may occur. The effects of hyperglycemia on the body are inherently slower and less of a problem from an acute (immediate) stand point. For these reasons, a quicker response to falling glucose in comparison to rising glucose may be desirable. Such a quick response may be accomplished by gain scheduling so that the response to rising and falling glucose levels are different. There are many possible methods of employing such gain scheduling. One such method defines zones using inverse polynomial curve fitting, as shown in FIG. 7.

In FIG. 7, each zone indicates the need for a specific action by the algorithm. For example, let us assume that the current glucose concentration is 160 mg/dl. If the goal is 100 mg/dl, then the PE=60 mg/dl (and the X axis, PE+40, is 100 mg/dl). If the glucose concentration is rising and the specific DE is 2, then the data pair falls into the green zone. Data within the green zone indicates that the computed insulin infusion rate (R) will not be altered (since there is a very low risk for hypoglycemia). If on the other hand, the PE remains at 60 (PE+40=100) and the DE is equal to −2, the data fair falls into the orange zone, indicating that there is a risk for hypoglycemia. Data pairs in the orange zone indicate that the Z_{PE} coefficient must be multiplied by a factor (chosen in one embodiment to be 2), thereby reducing the historical contribution, which in turn reduces the sum of the PE′ terms, which then reduces the overall infusion rate R.

Another illustrative example of gain scheduling is one in which PE remains unchanged and DE is −4. This would be the case, for example, if glucose concentration were falling rapidly. The data pair in this case falls into the red zone. Data pairs in the red zone indicate that the Z_{PE} coefficient is multiplied by a greater factor (chosen in one embodiment to be 4), further reducing the historical contribution, and thus further reducing the overall infusion rate R. In the situation in which PE remains unchanged and DE is −6, the data pair falls into the Off zone, which means that the infusion rate R is immediately turned off.

Use of the zone diagram illustrated in FIG. 7, which utilizes gain scheduling, is a cautionary measure to respond quickly to declining glucose values during closed loop control in order to reduce the risk for hypoglycemia. Persons skilled in the art will understand that other, similar methods of gain scheduling can be utilized with an FMPD algorithm.

For the algorithm as described above, if the glucose level remains at the goal, there is no proportional and no derivative error, and therefore no insulin will be infused. Let us assume that the goal is set at 100 mg/dl. It is known that the normal pancreatic islet cells continue to secrete insulin even thought glucose concentration may be equal or below 80 mg/dl. So, if the glucose goal is set at a level above the normal set point of the pancreatic islet cells, a basal insulin infusion rate may be added to the algorithm. If the goal is set a lower value and is similar to the true pancreatic set point, a separate basal insulin infusion will not be needed.

### Example 3

### Comparison to Other Algorithms

The use of proportional error (PE) and derivative error (DE) terms as used in glucose control has been discussed by others for use in an artificial pancreas, and algorithms incorporating these two errors have been termed proportional derivative (PD) algorithms. For example, a glucose-controlled insulin infusion system incorporating a PD method is disclosed in U.S. Pat. No. 4,151,845 to Clemens.

Glucose history may be incorporated into an algorithm for determining a response by using a Proportional, Integral, Derivative (PID) method, which incorporates an integral term into the algorithm. Steil, et al. disclosed the use of a PID algorithm in an artificial pancreas in U.S. Pat. No. 6,558,351. The PID algorithm can be summarized as follows. Assume that in the situation of serial glucose measurements, one plots the proportional error (current glucose concentration minus the glucose goal) on the ordinate and the time over which the measurements were made on the abscissa. The area under the curve from time x to time y is the integral, and this term provides some information about the history of the glucose values.

The FMPD algorithm of the present disclosure does not incorporate an integral term in the algorithm, and can be distinguished from PID algorithms quite readily. In FMPD algorithms, a time-weighting method is used for the analyte proportional error and the analyte derivative error. For both the proportional error and the derivative error, analyte values that are more recent are weighted more heavily than more remote values, and the degree to which more recent values are weighted more heavily than more remote values can be varied. In other words, the algorithm can be made to increase its response to prior events (but never so much that it responds to remote data more than more recent data).

In the specific context of an artificial pancreas, the FMPD algorithms of the present disclosure also can be compared to a PID algorithm in terms of how each reflects the normal physiology of pancreatic islet function. In terms of designing a closed loop artificial pancreas algorithm, it should be emphasized that the normal islet response to glucose comes to a plateau over time despite the presence of continued steady hyperglycemia. For example, in perifused islets and in non-diabetic humans who undergo hyperglycemic glucose clamps, insulin secretion typically begins to plateau within a two hour period despite a continued elevation of glucose. The time-related decrease in response is somewhat dependent on the degree of hyperglycemia; there may be less of a plateau with marked hyperglycemia. At any rate, after many hours, there is little or no continued rise in insulin secretion despite the persistence of hyperglycemia.

In an artificial pancreas system based on a PID algorithm, the integral factor responds to the duration of glucose elevation in a linear manner. That is, the magnitude of the insulin delivery rate is directly proportional to the length of time that the glucose concentration remains elevated. In a PID system, if glucose remains elevated at a constant level, the integral component will continue to rise in a linear fashion, rather than reach a plateau.

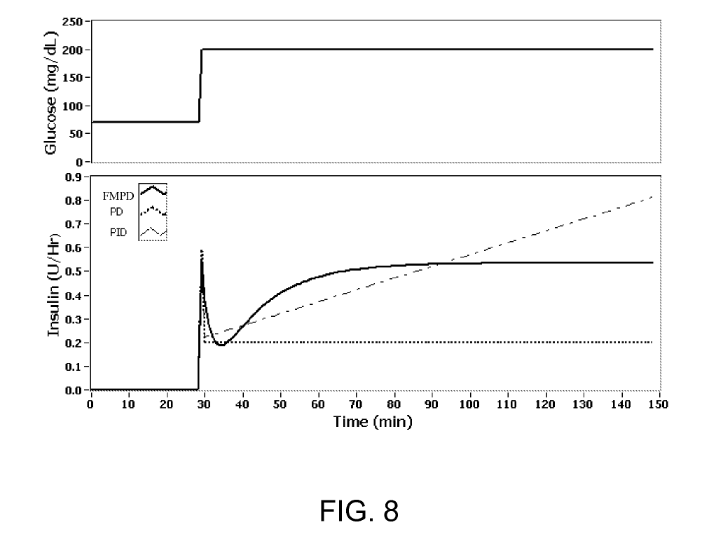

To compare the pancreatic response modeled by proportional derivative without fading memory (PD), PID without fading memory, and FMPD algorithms, we performed computations which simulated a hyperglycemic glucose clamp. The glucose values at every minute of the clamp profile were submitted to three algorithms: PD, PID and FMPD. The resulting insulin responses are shown in FIG. 8, which demonstrates that, like the normal physiologic response, all three algorithms demonstrate a biphasic response to elevated glucose. For both the PD and the PID algorithms, the first phase exists only at the instant when glucose level changed.

More specifically, the FMPD algorithm produces a first phase response that persists even after the instantaneous glucose rise. For the PD algorithm, the second phase insulin release is constant (unlike the normal situation, it does not rise). The PID algorithm produces a more realistic second phase in which the insulin infusion rate rises; however, the PID second phase continuously ramps up for the duration of the elevation of glucose concentration. This is because the integral action continues to add to the total insulin dose for as long as the glucose is above the set-point. The fading memory algorithm also produces an increasing second phase, but it reaches a plateau after a period of time, depending on the magnitude of the W_{PE} parameter. The FMPD algorithm in the present invention simulates the physiological situation of reaching a plateau by applying a fading memory of glucose data to the proportional and derivative components. The invention is based on the fact that the islet's physiological response to glucose is based on current information in addition to a fading memory of previous information.

### Example 4

### Avoiding Overcorrection Hypoglycemia

In another variation of the described methods, glucagon, or another substance capable of increasing glucose levels, is administered as glucose levels fall to avoid or attenuate hypoglycemia. As described above, insulin is delivered (intravenously, subcutaneously or intraperitoneally) based on the proportional error, the derivative error, as modified based on history (past proportional and derivative error calculations), which we refer to as “fading memory”.

In some instances if the glucose level starts out high, a relatively large dose of insulin is administered based on the algorithm. This may cause an overcorrection resulting in hypoglycemia several hours after giving the high dose of subcutaneous insulin. It is generally not practical or effective merely to turn off the insulin to avoid overcorrection hypoglycemia because subcutaneous insulin has a long delay before it is absorbed and its effect may last for hours after it is given.

Overcorrection hypoglycemia is typically not a problem when insulin is administered intravenously because its onset and offset is relatively rapid. However, when insulin is administered subcutaneously resulting in a rapid decline in glucose concentration, glucagon, or some other agent capable of increasing blood glucose levels (glucagon-like agent), or small volumes of concentrated glucose itself (for example 15-50% dextrose) may be adminstered subcutaneously.

Glucagon is an endogenous hormone that all mammals secrete from the pancreas. Glucagon is a linear peptide of 29 amino acids. Its primary sequence is almost perfectly conserved among vertebrates, and it is structurally related to the secretin family of peptide hormones. Glucagon is synthesized as proglucagon and proteolytically processed to yield glucagon within alpha cells of the pancreatic islets. Proglucagon is also expressed within the intestinal tract, where it is processed not into glucagon, but to a family of glucagon-like peptides (enteroglucagon).

In contrast to insulin, subcutaneous glucagon has a faster onset and offset. Studies have shown glucagon onset five to ten minutes after subcutaneous delivery, whereas insulin onset may take hours after subcutaneous delivery. Therefore it can be used effectively as a “rescue treatment” when the glucose level is declining rapidly. This has proven beneficial in animal studies to minimize overcorrection hypoglycemia.

For example, to avoid overcorrection hypoglycemia, glucagon, a glucagon-like agent, or some form of glucose itself, may be administered when blood glucose concentration is 100 mg/dl and falling rapidly. The calculation for dosing glucagon in a closed loop system may be similar to that of insulin, except in reverse. The amount of glucagon given may be based on the proportional error. Assume a set point of 100 mg/dl, more glucagon may be delivered if the glucose level were 60 than if the glucose level were 90. The amount of glucagon delivered may also be based on the derivative error (, e.g., the goal of the derivative error may be 0 or flat). In other words, if glucose were declining at 6 mg/dl per min, then one may give more glucagon than if it were declining at 1 mg/dl per minute.

The fading memory factor may be less important relative to the derivative error for glucagon administration, but may be more useful relative to the proportional error. Therefore, a fading memory calculation may be used, as described with respect to insulin delivery, for administering glucagon, mainly with respect to the proportional error, while considering none of the history of the derivative error or only a short history relative to the derivative error. In other words, it may be useful to consider a longer history for the proportional error than for the derivative error.

For example, assume patient A has had a glucose concentration slope of 1 for at least forty five (45) minutes, and currently has a glucose concentration of 80 mg/dl; and patient B has had a glucose concentration slope of 1 for only a short period, and currently has a glucose concentration of 80. Patient A has a higher risk of hypoglycemia than patient B, but perhaps not a lot higher. Both patients have a glucose concentration falling at the same rate, and both patients are nearing hypoglycemia. However, if the glucose level (proportional error) is unchanged compared to thirty minutes ago when glucagon was administered, then more glucagon should be administered immediately.

### IV. EXAMPLES OF GLUCAGON ADMINISTRATION

As explained above, glucagon may be given when glucose is falling and approaching hypoglycemic levels. In a method of glucose control based on a proportional derivative algorithm, the amount of glucagon called for by an algorithm may be based on (a) the difference between the target glucose value and the present glucose value (the proportional error) and (b) on the rate of decline of glucose (the derivative error). The infusion of glucagon may be initiated when the proportional error and the derivative errors exceed given criteria. For example, if glucose is below the target and falling rapidly, glucagon may be given.

Some systems of low-level glucose correction in accordance with the present disclosure, may include a fluid delivery period, also referred to as a pulse, bolus, infusion period and/or dose, of a glucose increasing substance, such as glucagon, followed by a refractory period. In some embodiments, the duration and/or amount of fluid delivered by or during the fluid delivery period may be determined in part by a pre-determined maximum fluid delivery duration and/or a pre-determined maximum fluid delivery amount. Additionally and/or alternatively, the fluid delivery period may be at least partially dependant on the body weight of a subject (e.g., user).

The refractory period may include a period of time, after the fluid delivery period, during which the system does not allow further administration of the fluid. The refractory period may be dependant at least in part on the duration of and/or amount of fluid delivered in the fluid delivery period. For example, the duration of the refractory period may be dependant on the duration of the fluid delivery period, (e.g., 1.5 times longer than the fluid delivery period).

With respect to glucagon delivery, the refractory period is important because if too much glucagon is delivered, there is risk of depleting glycogen, an animal starch stored in the liver that can be broken down to generate glucose. If glycogen is depleted, then the body is unable to respond to glucagon. In such a case, the individual is at risk for developing severe hypoglycemia. The refractory period is also helpful in avoiding large doses of glucagon that can lead to side effects such as nausea or vomiting.

A glucagon administration method within a closed loop diabetes control system in accordance with the present disclosure may include a short-lived, brisk pulse of glucagon given under the skin or by another route during incipient hypoglycemia. The amount and/or duration of the pulse of glucagon may depend in part on a pre-determined maximum glucagon amount and/or a pre-determined glucagon delivery duration. The amount of the pulse of glucagon may be dependant on a meter squared of body surface area measurement and the duration may be dependant on a pre-determined maximum. After each pulse of glucagon, there is a refractory period during which glucagon cannot be given. In this way, the patient will obtain the benefit of an early infusion of a brisk dose of glucagon, which rapidly stimulates the liver to make glucose from glycogen, thus preventing hypoglycemia.

For example, the glucagon pulse may be at least 16 micrograms [mcg] of glucagon per meter squared of body surface area (or 0.4 mcg per kg body weight), though in some cases, smaller doses may be called for. The pulse may be given over a period having a pre-determined maximum, for example a maximum time period of 15 minutes. The refractory period that follows this pulse may be at least 1.5 times the duration of the infusion. For example, if the infusion is given over 10 minutes, the refractory period may be at least 15 minutes (or longer).

As described above, in some embodiments, there may be a pre-determined maximum amount of glucagon that can be given over the infusion period. The pre-determined maximum may be at least partially dependant on meter squared of body surface area and/or body weight. In some embodiments, the maximum may be no greater than 96 mcg per meter sq, which is approximately equal to 2.4 mcg per kg of body weight. In other embodiments, smaller maximums are effective. In other embodiments, larger maximums are effective. If the dose of the glucagon pulse is allowed to exceed the maximum limit, there is a risk for rapid depletion of liver glycogen and side effects such as nausea or vomiting can occur.

The term “front-loaded” and/or “front-loaded fluid delivery” may be used to describe one or more doses of fluid and/or glucose increasing substance, with each dose being followed by a refractory period. This method of front-loaded glucagon delivery, with a refractory period, is applicable to glucagon delivery methods having proportional-derivative algorithms, proportional-integral-derivative algorithms, model predictive control algorithms, neural network algorithms, fuzzy logic algorithms, and other predictive and/or control algorithms.

For example, in embodiments of a glucagon delivery method at least partially dependant on a proportional-derivative algorithm, in order to deliver sufficient amounts of glucagon to consistently raise the glucose level of a subject, it may be necessary to use high (more negative) gain settings for the proportional and derivative factors. The gain settings may be configured to rapidly increase a subject's glucose level. In some embodiments, the proportional gain factor may be higher (more negative) than the derivative gain factor. However, if the algorithm continuously uses these high gain settings (with no refractory periods), the patient might receive too much glucagon and thus deplete liver glycogen. The refractory period allows the intermittent use of high gain proportional and derivative factors without a risk for glycogen depletion.

An automated front-loaded glucagon delivery may be used as part of a closed loop diabetes control system wherein there is bi-hormonal control (control via insulin deliver and glucagon delivery). For example a closed loop control system consisting of one or more glucose-measuring devices or sensors, for example a first sensor and a second sensor from which data are collected and/or compared to determine the most accurate sensed glucose level. The most accurate sensed data is then entered into an algorithm, which in turn controls insulin delivery to a subject, for example as explained above using a fading memory algorithm or by any other means known in the art.

In such embodiments, glucagon may also be administered as part of the closed loop control system, as controlled by a front-loaded fluid delivery algorithm. For example, insulin and glucagon may be delivered via a dual chambered pump or each administered fluid may have distinct pumps. The front loaded glucagon delivery may have a pre-determined limit per pulse, for example a limit of no greater than 96 mcg per meter sq, which is approximately equal to 2.4 mcg per kg of body weight. In other examples, a lower limit will suffice. A refractory period may be dependant on the glucagon infusion period, in order to limit the total amount of glucagon delivered to the patient. For example, the refractory period may be at least 1.5 times as long as the glucagon infusion period.

The first two examples presented below demonstrate differing patterns of glucagon delivery in a patient with Type 1 diabetes. The first example has no front-loading and no refractory period. The second example has front-loading and a refractory period.

### Example 1

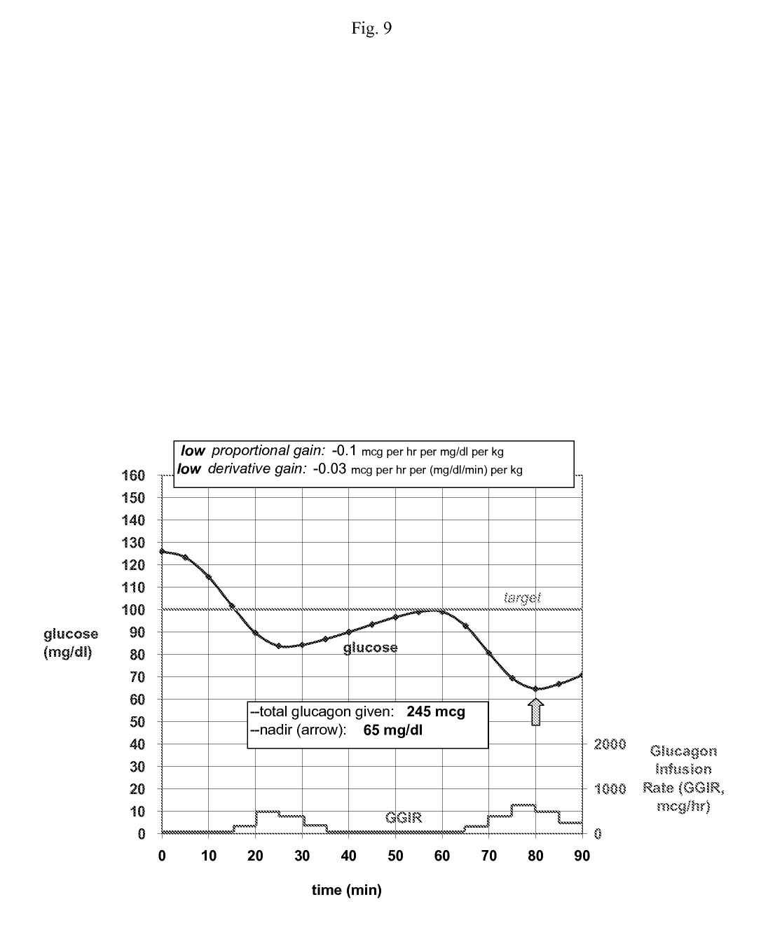

In the first example a proportional derivative algorithm is used and explained with reference to FIG. 9. The gain factors that control the dose of glucagon are low. The proportional gain is −0.1 and the derivative gain is −0.03. There is no maximal dose limit and there is no refractory period. In other words, the algorithm operates the same way continuously throughout this 90 min period. The action of insulin is set to be large and identical in the two examples. Note that in Example 1, the algorithm calls for a slow infusion of glucagon at approximately minute 15 and this infusion continues until minute 35. A second infusion is called for at minute 70 and is continuing at the end of the 90 minute point. Glucose values are shown in the upper curve with symbols and the glucagon infusion rate is shown as solid straight lines in the lower part of the graph.

Note that in this example, the second infusion of glucagon is not successful: the glucose falls to a value of 65 mg/dl, which constitutes a bona-fide episode of hypoglycemia. A patient experiencing this blood glucose value would likely feel poorly, and might well have tremor, sweating and confusion.

### Example 2

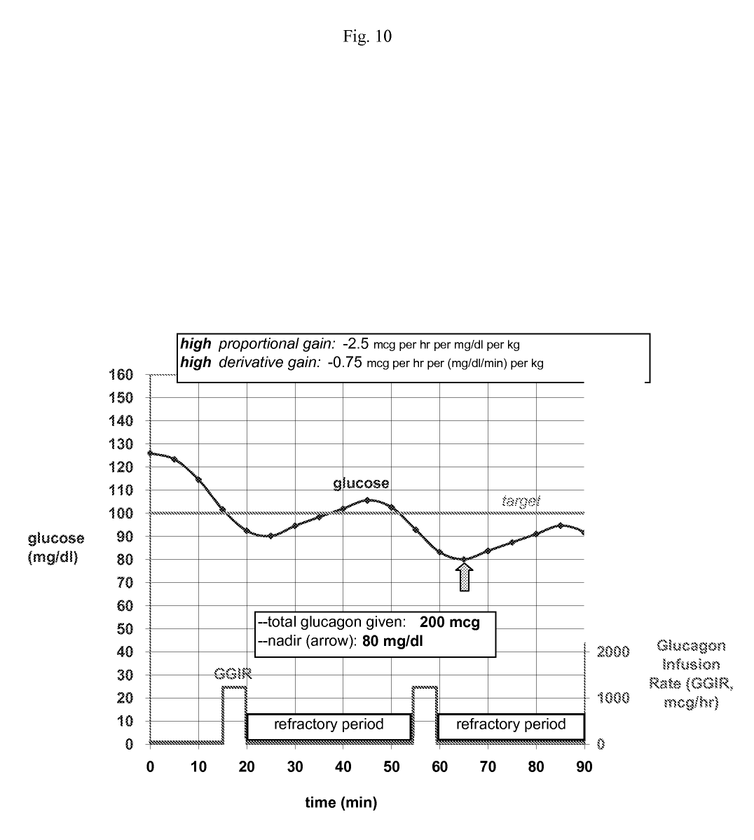

In the second example, a front-loaded proportional derivative algorithm having a refractory period is used and explained with reference to FIG. 10. In this example, the gain factors are set at greater magnitudes (more negative) than in Example 1 (FIG. 9). The proportional gain is set at −2.5 and the derivative gain is set at −0.75. Glucose starts out at 125, just as in the first example, and the insulin effect is identical to the first example. In this example, there is a 35 minute refractory period that is initiated after the delivery of each glucagon pulse of 100 mcg. Note that as glucose declines, at minute 15, the algorithm calls for a brisk pulse of glucagon which is given over a 5 minute period, from minute 15 to minute 20. Over this period, the maximal amount of glucagon for this example (100 mcg) is given. The brisk pulse leads to the intended effect, a rise in glucose. Later during this simulation, from minute 55-60, the algorithm once again calls for an infusion of glucagon and again, 100 mcg (the limit) is given. In this case, the short, brisk delivery of glucagon is once again successful and the glucose nadir (trough) is only 80 mg/dl, a normal level that would not lead to symptoms.

It is also important to note that the total glucagon dose in Example 2, in which the more successful algorithm was employed, was actually less than the dose in Example 1. The total dose over 90 minutes was 200 mcg in Example 2 vs 245 mcg in Example 1. Thus, the scenario in Example 2 was not only more successful in avoiding hypoglycemia but also had a lesser risk for depleting liver glycogen.

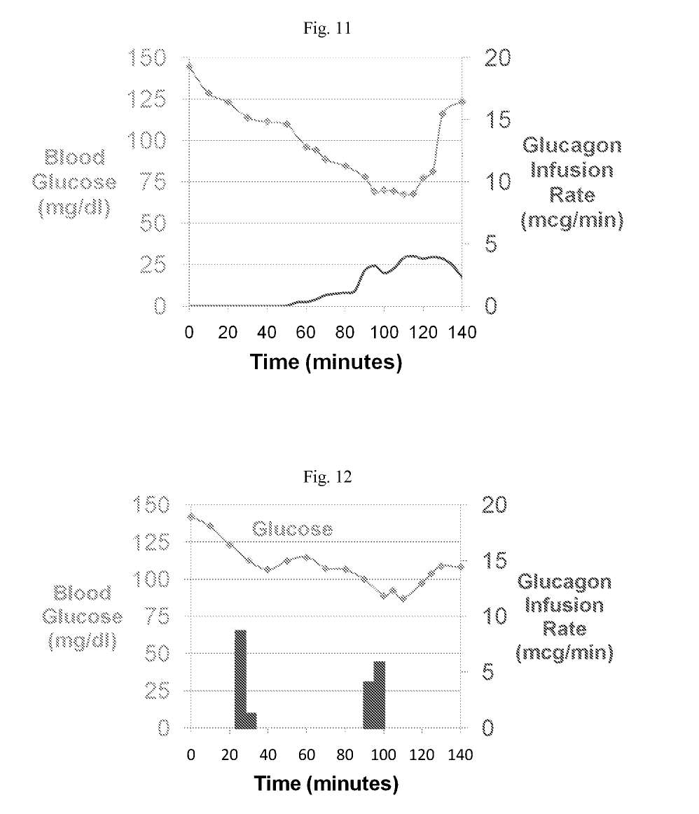

Examples 3 and 4 presented below, and explained with reference to FIGS. 11-16, demonstrate a comparison of insulin delivery alone and insulin plus glucagon delivery at different rates in patients with type 1 diabetes. In the comparison, 9 subjects with type 1 diabetes took part in 12 studies (9 or 28 h). Each subject underwent both study conditions of aspart insulin (Novo Nordisk A/S of Denmark) alone and insulin in combination with glucagon, wherein the glucagon is administered for low/falling glucose modified by a fading memory of glucose history.

The system used in the examples 3 and 4 was a sensor-controlled system, having dual sensors. The more accurate sensor was used to control insulin/glucagon delivery rates. The system used was a hybrid system, wherein the pre-meal insulin was 40-75% of typical dose, and included insulin on board (IOB). Venous glucose was measured every 10 minutes for the duration of the studies to provide ongoing assessment of sensor accuracy. Estimated insulin on board was continually calculated, which correlated to the free insulin plasma levels, and insulin infusion was temporarily discontinued when 10B reached a pre-determined threshold.

### Example 3

In example 3, and as shown in FIG. 11, glucagon is administered via a prolonged infusion as determined by a fading memory proportional derivative algorithm having low gain factors. FIG. 11 is an example of one of the subjects that received glucagon with a lower gain and over a more prolonged period, and this subject developed overt hypoglycemia with a nadir blood glucose of 68 mg/dl, and required treatment with oral carbohydrate.

### Example 4

In example 4 and as shown in FIG. 12, glucagon is administered via a brief (front-loaded) infusion as determined by a front-loaded fading memory proportional derivative algorithm having high (more negative) gain factors and a refractory period following each fluid delivery period that limits total amount delivered.

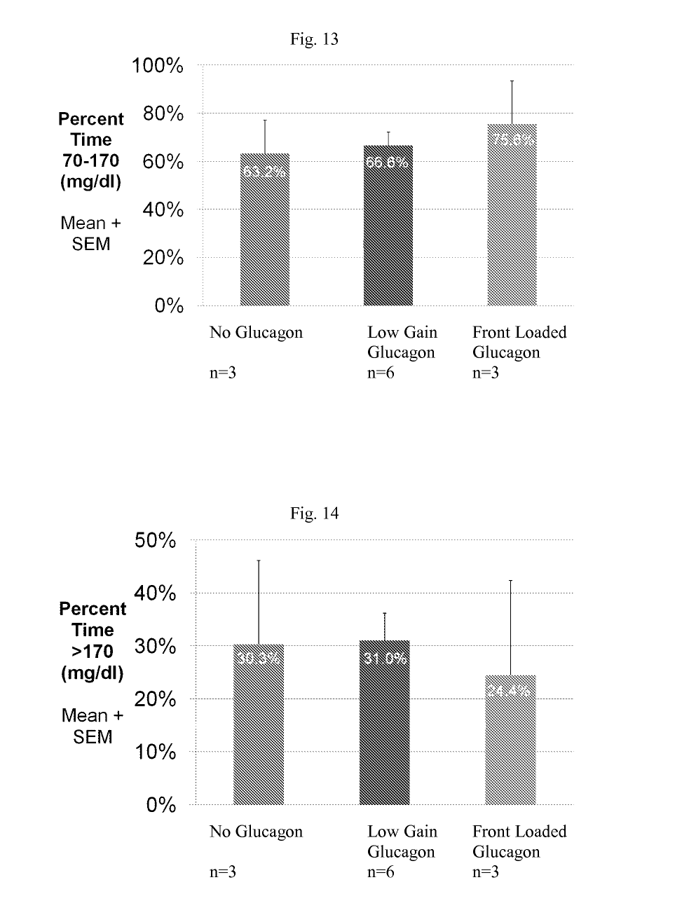

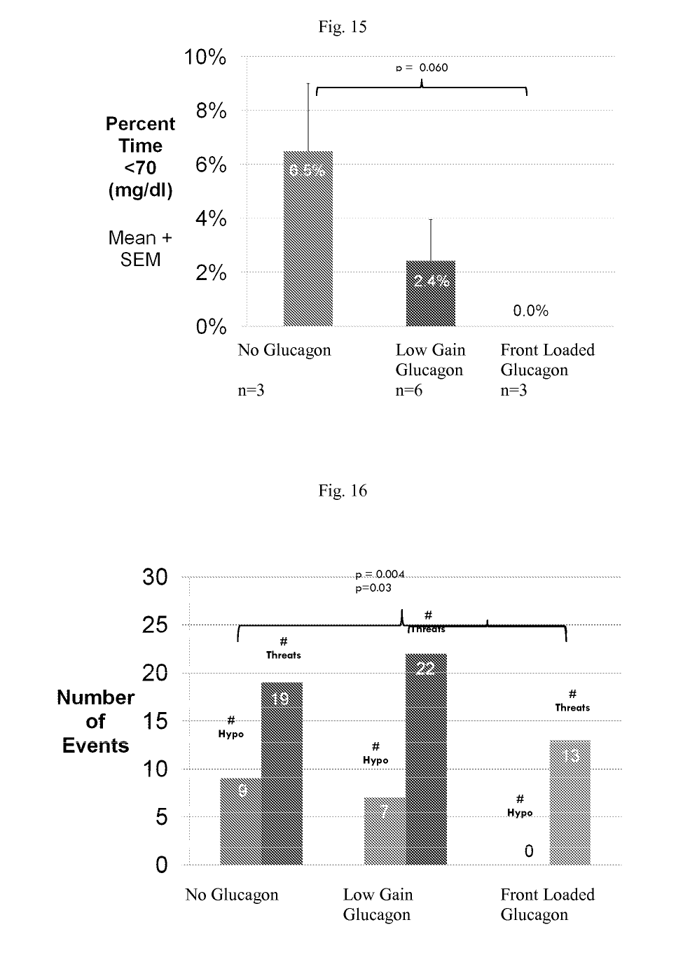

FIG. 13 shows daytime results, time in target range, wherein the front-loaded glucagon infusion having a refractory period is shown to have the largest percentage of time in the target range. FIG. 14 shows time in Hyperglycemic Range, wherein the front-loaded glucagon infusion having a refractory period is shown to have the lowest percentage of time in the hyperglycemic range. FIG. 15 shows time in Hypoglycemic Range, wherein the front-loaded glucagon infusion having a refractory period is shown to have the lowest percentage of time in the hypoglycemic range.

FIG. 16 shows a Prevention of Hypoglycemia chart. Different subjects have varying risks for hypoglycemia. FIG. 16 shows the number of hypoglycemic threats that resulted in overt hypoglycemia in each of the three conditions. A threat is a situation in which hypoglycemia, defined as blood glucose less than 70, will occur within 40 minutes if the present glucose slope remains unchanged.

The data from Examples 3 and 4 suggest front-loaded glucagon reduces the risk of hypoglycemia in subjects with type 1 diabetes in a closed loop system compared to low gain glucagon and compared to insulin alone.

### Example 5

In this example, to minimize hypoglycemia in subjects with type 1 diabetes by automated glucagon delivery in a closed-loop insulin delivery system, adult subjects with type 1 diabetes underwent one closed-loop study with insulin plus placebo and one study with insulin plus glucagon, given at times of impending hypoglycemia. As discussed in further detail below, and in Novel Use of Gucagon in a Closed-Loop System for Prevention of Hypoglycemia in Type 1 Diabetes, e-published Mar. 23, 2010, http://care.diabetesjournals.org on March 23, Castle et al., and incorporated by reference in its entirety, seven subjects received glucagon using high-gain parameters, and six subjects received glucagon in a more prolonged manner using low-gain parameters. Blood glucose levels were measured every 10 min and insulin and glucagon infusions were adjusted every 5 min. All subjects received a portion of their usual premeal insulin after meal announcement. Delivery of insulin and glucagon was automated and controlled by an amperometric glucose sensor.

Glucagon plus insulin delivery, compared with placebo plus insulin, significantly reduced time spent in the hypoglycemic range (15±6 vs. 40±10 min/day, P=0.04). Compared with placebo, high-gain glucagon delivery reduced the frequency of hypoglycemic events (1.0±0.6 vs. 2.1±0.6 events/day, P=0.01) and the need for carbohydrate treatment (1.4±0.8 vs. 4.0±1.4 treatments/day, P=0.01). Glucagon given with low-gain parameters did not significantly reduce hypoglycemic event frequency (P=NS) but did reduce frequency of carbohydrate treatment (P=0.05).

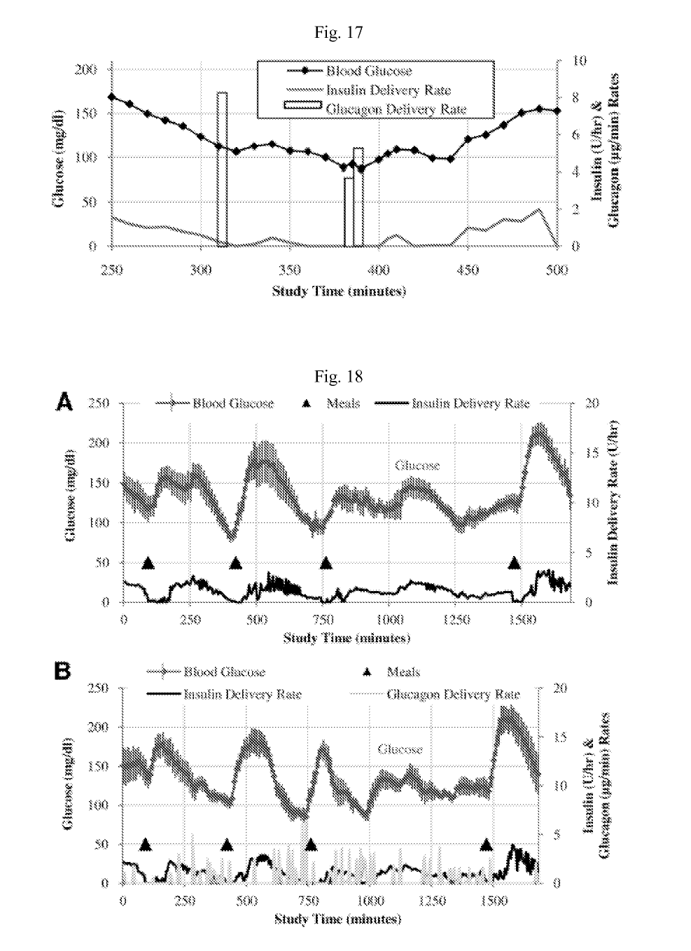

FIG. 17 is an example of data taken from a closed-loop study. Venous blood glucose is noted by black diamonds, insulin delivery rate by a gray line, and glucagon delivery rate by rectangles. Note that glucagon is delivered by algorithm in the late postprandial period at times of impending hypoglycemia. Overt hypoglycemia is avoided without the use of carbohydrate supplementation.

FIG. 18A-B is a summary of glucose levels (means±SE), insulin delivery rate, and, for glucagon studies, the glucagon delivery rate. Venous blood glucose is noted by gray diamonds, insulin delivery rate by a black line, glucagon delivery rate by a light gray line, and meals by black triangles. A: Composite of eight insulin plus placebo studies. B: Composite of seven insulin plus high-gain glucagon studies. Insulin delivery and overall glycemic control were similar in both conditions.

In the example, subjects wore two subcutaneous glucose sensors, either Seven Plus, sold by DexCom, Inc. of San Diego, Calif., or Guardian Real-Time, sold by Medtronic Diabetes of Northridge, Calif., glucose sensors. Sensors were placed 8-24 h prior to beginning the study. For subjects taking long-acting insulin at night, the dose was reduced by 50% the night prior to the study. An intravenous catheter was placed in a forearm vein. The forearm was warmed with a heating pad to arterialize the venous blood. Venous glucose was measured every 10 min in duplicate using HemoCue Glucose 201 Analyzer, sold by HemoCue, Inc of Lake Forest, Calif. Glucose sensor readings were recorded from the receivers every 5 min. For the first 2 h, the insulin and glucagon delivery rates were determined by venous glucose levels. After the first 2 h, the sensed glucose values from the sensor with better accuracy were input into the algorithm every 5 min to determine the hormone delivery rates. If the sensor accuracy became suboptimal, defined as a median absolute relative difference (MARD) exceeding 20% or median absolute difference (MAD) exceeding 20 mg/dl, control was switched to the other sensor. If the accuracy of both sensors was poor, control was switched to venous glucose and the sensors were recalibrated. Sensors were calibrated at a minimum of every 12 h.

The Fading Memory Proportional Derivative (FMPD) algorithm, as described above, was used to determine the insulin and subcutaneous glucagon (or placebo) delivery rates. Aspart insulin (Novo Nordisk NS of Denmark) was delivered subcutaneously via an Animas IR 1000 insulin pump (Animas Corporation of West Chester, Pa.). Glucagon or saline placebo was given through a subcutaneous catheter via a Medfusion 2001 syringe pump (Smiths Medical of Dublin, Ohio). One milligram of glucagon (Novo Nordisk NS of Denmark) was mixed with 3 ml of sterile water. The glucagon preparation was freshly reconstituted every 8 h. The insulin delivery rate and glucagon delivery rate was adjusted every 5 min, based on the controller output. The FMPD algorithm determined the hormone delivery rates based on proportional error, defined as the difference between the current glucose level and the target level, and the derivative error, defined as the rate of change of the glucose. The “fading memory” designation refers to weighting recent errors more heavily than remote errors. This weighting provides an adaptive component to the algorithm, as described above. Basically, the insulin rate was increased for high or rising glucose levels and glucagon was given for low or falling glucose levels. The basal insulin infusion rate (in units per hour) was given at a rate of 35% of the patient's typical total daily insulin dose, divided by 24.

Determination of Insulin Delivery

In the FMPD algorithm, the gain factors determined the degree to which proportional or derivative errors led to changes in hormone delivery rates. There were separate gain factors for insulin and glucagon. Positive proportional errors (glucose level above target) and positive derivative errors (rising glucose level) called for an increase in the insulin delivery rate. The overall insulin delivery rate was determined by adding the rates called for by the proportional error (IIRₚₑ), the derivative error (IIR_{de}), and the basal insulin rate. The proportional error gain factor was 1.2×10⁻³±0.078×10⁻³ units/kg per mg/dl/h for glucagon studies and 1.3×10⁻³ units/kg per mg/dl/h for placebo studies. The derivative error gain factor was 2.0×10⁻³±0.096×10⁻³ units/kg per mg/di for glucagon studies and was 2.0×10⁻³ units/kg per mg/di for placebo studies. The mean blood glucose target was 110±1 mg/di for glucagon studies and 110 mg/di for placebo studies. There were no significant differences between any of these parameters between the groups. For subjects who under went two closed-loop studies, the algorithm parameters were identical for both.

Insulin on board, the amount of insulin that had been delivered and was assumed to be active, was continually estimated using methods known to those skilled in the art. To minimize hypoglycemia, the insulin infusion was discontinued if the estimated insulin on board reached 15% of the subject's estimated total daily insulin requirement.

The proportional and derivative error gain factors for glucagon were negative, such that negative proportional and derivative errors called for an increase in the glucagon rate. For glucagon, the average weighted proportional error was calculated over a 15-min interval and the average weighted derivative error was calculated over a 10-min interval. There was no basal glucagon infusion rate. In this example, two closely related algorithms were tested for administering glucagon. Four subjects completed 9-h studies and two subjects completed 28-h studies with low-gain factor settings. In these low-gain glucagon studies, the mean proportional error gain factor was −0.23±0.04 ml/kg per mg/dl/h, the mean derivative error gain factor was −0.06±0.009 ml/kg per mg/dl, and target glucose for glucagon infusion was 108±3 mg/dl.

Two subjects completed 9-h studies and five subjects completed 28-h studies with high-gain factor settings. For all of these high-gain glucagon studies, the proportional error gain factor was −2.70 ml/kg per mg/dl/hour, the derivative gain factor was −0.60 ml/kg per mg/dl, and the target glucose for glucagon infusion was 97±1 mg/dl. To avoid over delivery of glucagon, when total glucagon delivery over the prior 50 min reached a ceiling of 1.0 pg/kg, the algorithm initiated a refractory period for the subsequent 50 min, during which glucagon could not be delivered. Thus, short pulses of glucagon delivery over 5-10 min were followed by the absence of glucagon delivery for 50 min. The insulin rate was reduced by 75% for 40 min after each maximal glucagon pulse.

Patients were given two meals during each 9-h study and four meals during each 28-h study. Each meal was announced to the controller and an open loop pre-meal bolus was given. Aspart insulin was given 0-10 min before meals, depending on the subject's premeal glucose level. For low-gain glucagon studies, 53.3±7.0% of usual premeal insulin dose was given. The amount of premeal insulin was increased after the first four studies because of a pattern of postprandial hyperglycemia in those studies. For all placebo and high-gain glucagon studies, 75% of the usual premeal insulin dose was given.

Subjects were treated for hypoglycemia if the venous glucose value fell below 70 mg/dl. For glucose levels 60-69 mg/dl, subjects were given 15 g oral carbohydrate, and the treatment repeated as needed every 15 min. For a glucose value <60 mg/dl, 10 g dextrose was given intravenously.

Arterialized venous glucose values, not sensed glucose values, were used to compare hypoglycemia and glucose control between groups. Glucose area under the curve (AUC) was calculated using methods known to those skilled the state of the art. Minutes in the hypoglycemic range, defined as glucose <70 mg/dl; hypoglycemic events; treatments for hypoglycemia; units of insulin delivered; and micrograms of glucagon delivered were normalized to 24 h for data from both 9- and 28-h studies. Data are expressed as means±SE. Sensor accuracy was calculated by comparing sensor glucose to reference glucose values. Comparisons were made using paired or unpaired t tests, as appropriate. Calculations were performed using Microsoft Excel 2007 (version 12) (Microsoft Corporation of Redmond, Wash.).

Six women and seven men with type 1 diabetes participated in a total of 21 human closed-loop studies with a duration of 21.5±2.0 h. Seven subjects received glucagon delivered in a brisk fashion (high gain) and six subjects received glucagon delivered in a slower fashion (low gain). In both the high- and low-gain glucagon studies, glucagon was typically delivered at times of impending hypoglycemia when glucose was 90-120 mg/dl, depending on the rate of glucose decline (FIG. 17). At these times, insulin delivery was also markedly reduced or discontinued by the insulin algorithm. The high-gain glucagon results (paired analysis), low-gain glucagon results (unpaired analysis), and combined high- and low-gain glucagon results (unpaired analysis) are presented separately below. One subject who received high gain glucagon but did not return for a placebo study was included in the combined results but was not included in the paired high-gain analysis.

In six subjects who underwent both high-gain glucagon study and a placebo study, there was a 56% reduction in time spent in the hypoglycemic range (18±11 vs. 41±13 min/day, P=0.01). The number of hypoglycemic events, with events lasting >20 min being considered a new event, was also significantly reduced during the high-gain glucagon versus placebo studies (1.0±0.6 vs. 2.1±0.6 events/day, P=0.01), as was the number of oral or intravenous carbohydrate treatments for hypoglycemia (1.4±0.8 vs. 4.0±1.4 treatments/day, P=0.01). There was no significant difference in mean glucose between the high-gain glucagon versus placebo studies (138±17 vs. 131±17 mg/dl, P=NS), as shown in FIG. 18A. The mean fasting glucose was also quite similar (123±14 vs. 120±15 mg/dl, P=NS). There was a nonsignificant trend toward a higher postprandial glucose in high-gain glucagon versus placebo studies, defined as mean value 0-180 min after meals (157±18 vs. 144±17 mg/dl, P=NS). The amount of insulin delivered during the high-gain glucagon versus placebo studies was nearly identical (48.9±6.2 vs. 48.3±5.5 units per day, P=NS).

In six subjects who received low-gain glucagon compared with the eight subjects who received placebo, there was a nonsignificant reduction in time in the hypoglycemic range (15±8 vs. 40±10 min/day, P=NS). There was also a trend toward a reduction in the number of hypoglycemic events that did not reach statistical significance (1.4±0.7 vs. 2.3±0.5 events/day, P=NS). There was a reduction in the number of treatments for hypoglycemia in studies with low-gain glucagon of borderline significance (1.0±0.7 vs. 3.9±1.0 treatments/day, P=0.05).Mean glucose was somewhat higher in low-gain glucagon versus placebo studies (157±24 vs. 135±16 mg/dl, P=0.04). There was also a trend toward higher fasting glucose in the low-gain glucagon versus placebo studies (137±20 vs. 122±13 mg/dl, P=NS). There was a similar trend, of borderline statistical significance, suggesting a larger elevation in postprandial glucose in the low-gain glucagon versus placebo studies (179±26 vs. 151±18 mg/dl, P=0.05). There was a nonsignificant difference in insulin delivered in low-gain glucagon versus placebo studies (60.1±14.1 vs. 46.9±5.5 units/day). The mean dose of glucagon delivered during the low-gain glucagon studies was higher than the high-gain glucagon studies but did not reach statistical significance (746±134 vs. 516±108 μg/day, P=NS).

Glucagon, when given either via high or low gain, compared with placebo, led to a 63% reduction of time spent in the hypoglycemic range (15±6 vs. 40±10 min/day, P=0.04). The number of hypoglycemic events per day was not significantly different between glucagon versus placebo studies (1.1±0.4 vs. 2.3±0.5 events/day, P=NS). The number of treatments for hypoglycemia per day was considerably reduced in the glucagon versus placebo studies (1.1±0.5 vs. 3.9±1.0 treatments/day, P=0.01). Mean glucose was somewhat higher in the glucagon studies, but this increase did not reach statistical significance (145±14 vs. 135±16 mg/dl, P=NS). Other metrics of glycemic control, including percent of AUC in the target (70-180 mg/dl) and hyperglycemic (>180 mg/dl) ranges and mean amplitude of glycemic excursions were not significantly different between the groups (data not shown).

In this automated glycemic control system, the effect of subcutaneous glucagon, delivered in small doses at times of impending hypoglycemia, was compared to saline placebo. In both conditions, the algorithm called for a significant reduction or discontinuation of insulin delivery during impending hypoglycemia. Compared with placebo, glucagon delivered in pulses using high-gain parameters significantly decreased the time spent in the hypoglycemic range, the number of hypoglycemic events, and the number of treatments needed for hypoglycemia. Only the high-gain, not the low-gain, glucagon delivery system was superior to placebo in reducing all three of these outcomes, despite the fact that a lower amount of glucagon was delivered in the high-gain studies. The high-gain glucagon infusion consisted of a pulse of glucagon typically given over 5-10 min at a time of impending hypoglycemia followed by a 50-min off period. The low-gain glucagon was delivered in a slow, more prolonged manner without a mandatory off period. The high-gain glucagon infusion may be more physiologic, as glucagon is secreted rapidly in response to hypoglycemia in humans without diabetes. Minimizing glucagon delivery, as described here, may be important to avoid potential side effects, such as acute hyperglycemia and nausea, and more severe effects, such as depletion of liver glycogen. Notably, the mean glucose levels in the high-gain glucagon and placebo studies were very similar. These results present how an automated system of closed-loop glucagon delivery, with hybrid pattern of insulin delivery including meal announcement, is able to control glycemia safely and effectively in people with type 1 diabetes.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

I claim:

1. An infusion system for infusing a fluid into a subject, comprising:
a first sensor configured to monitor concentration of an analyte in the subject and to generate a sensor output signal based on the monitored concentration, wherein the sensor output signal is used to generate a processor input signal;
a front-loaded fluid delivery processor configured to process the processor input signal and to generate a processor output signal having a fluid delivery period and a refractory period; and
a fluid delivery system configured to infuse fluid into the subject in a manner based at least partially on the processor output signal.

2. The infusion system of claim 1, wherein the analyte is glucose.

3. The infusion system of claim 1, wherein the fluid contains a glucose-increasing substance.

4. The infusion system of claim 1, wherein the processor includes a proportional derivative algorithm having a high proportional gain factor and a high derivative gain factor.

5. The infusion system of claim 1, wherein the first sensor includes a disc-shaped body having two opposing sides, a cathode and a plurality of anodes positioned on at least one of the sides.

6. The infusion system of claim 1, further comprising a membrane semi-permeable to the analyte covering at least a portion of the first sensor.

7. The infusion system of claim 6, further comprising an enzyme layer disposed between the first sensor and the membrane.

8. The infusion system of claim 1, further comprising a transmitter electrically coupled to the first sensor, wherein the transmitter is configured to receive the sensor output signal, to convert the first sensor output signal into the processor input signal, and to transmit the processor input signal to the processor.

9. The infusion system of claim 1, wherein the processor output signal is at least partially dependant on the body weight of the subject.

10. The infusion system of claim 1, wherein the fluid delivery period is at least partially dependant on at least one of a pre-determined maximum fluid delivery amount and a pre-determined maximum fluid delivery duration.

11. The infusion system of claim 1, wherein the duration of the refractory period is at least partially dependant on the fluid delivery period.

12. The infusion system of claim 1, further comprising a second sensor configured to monitor concentration of the analyte in the subject and to generate a sensor output signal based on the monitored concentration.

13. The infusion system of claim 3, wherein the fluid delivery system is configured to infuse the fluid containing the glucose-increasing substance into the subject and a fluid containing insulin into the subject.

14. A method of determining a fluid infusion rate for infusing a fluid into a subject, comprising:
monitoring concentration of an analyte in the subject;
generating a sensor output signal based on the monitored concentration;
generating a processor input signal from the sensor output signal;
processing the processor input signal using a front-loaded fluid delivery algorithm to generate a processor output signal having a fluid delivery period and a refractory period following the fluid delivery period; and
determining the fluid infusion rate based at least partially on the processor output signal.

15. The method of claim 14, further comprising infusing the fluid into the user at the determined fluid infusion rate.

16. The method of claim 14, wherein the analyte is glucose.

17. The method of claim 16, wherein the fluid contains a glucose-increasing substance.

18. The method of claim 17, further comprising delivering the fluid containing the glucose-increasing substance into the subject.

19. The method of claim 14, wherein monitoring concentration of an analyte in the subject includes at least a first sensor and a second sensor and the more accurate sensor output signal is used in generating a processor output signal.

20. The method of claim 16, further comprising
delivering insulin to the subject when the subject's glucose concentration is rising at a rate above a pre-determined threshold, and the subject's glucose concentration has risen above a pre-determined threshold level.

21. The method of claim 17, wherein the glucose-increasing substance is selected from the following: (a) glucagon, (b) glucagon-like agent, (c) glucose, and (d) dextrose, and mixtures thereof.

22. The method of claim 14, further comprising receiving the sensor output signal at a transmitter electrically coupled to the sensor, converting the sensor output signal to the processor input signal with the transmitter, and transmitting the processor input signal to the processor with the transmitter.

23. The method of claim 14, wherein determining the fluid infusion rate is at least partially based on the body weight of the subject.

24. The method of claim 14, wherein determining the fluid infusion rate is at least partially based on at least one of the following: a pre-determined maximum fluid delivery amount for the fluid delivery period and a pre-determined maximum fluid delivery duration for the fluid delivery period.

25. The method of claim 14, wherein the refractory period has a longer duration than the fluid delivery period.

26. A method of controlling concentration of an analyte in a mammal, comprising:
repeatedly measuring concentration of an analyte in a mammal with a first sensor implanted in the mammal;
using a front-loaded algorithm to compute a fluid delivery rate based on the measured analyte concentration; and
delivering fluid to the mammal at the computed fluid delivery rate, the computed fluid delivery rate including a fluid delivery period and a refractory period.

27. The method of claim 26, wherein the fluid delivery period is limited by at least one of a maximum time period for fluid delivery and a maximum fluid delivery amount.

28. The method of claim 26, wherein the analyte is glucose and the fluid contains a glucose-increasing substance.

29. The method of claim 26, wherein delivering fluid to the mammal includes delivering a glucose-increasing substance to the mammal with a glucose-increasing substance delivery device located outside the body of the mammal.

30. The method of claim 26, wherein delivering fluid to the mammal includes delivering a glucose-increasing substance to the mammal with a glucose-increasing substance delivery device implanted inside the body of the mammal.

31. The method of claim 26, wherein the front-loaded algorithm computes the fluid delivery rate based on a proportional derivative algorithm having a high proportional error gain factor and a high derivative error gain factor.

32. The method of claim 26, further comprising repeatedly measuring concentration of an analyte in a mammal with a second sensor implanted in the mammal; and
using the more accurate measured analyte concentration from either the first sensor or the second sensor to compute the fluid delivery rate.

33. The method of claim 28, further comprising delivering insulin to the mammal when the mammal's glucose concentration is rising at a rate above a pre-determined threshold, and the mammal's glucose concentration has risen above a pre-determined threshold level.
